SEQUENCE LISTING (40 sequences)

SEQ ID NO: 1 - protein, 20 aa, Artificial Sequence
  Synthetic Peptide
  misc_feature (7)..(7): Xaa is a standard amino acid or a non-genetically-encoded amino acid
  misc_feature (9)..(12): Xaa is a standard amino acid or a non-genetically-encoded amino acid
  misc_feature (18)..(18): Xaa is a standard amino acid or a non-genetically-encoded amino acid
LRRFSTXPXXXXNINNVXNF

SEQ ID NO: 2 - protein, 20 aa, Artificial Sequence
  Synthetic Peptide
  MISC_FEATURE (7)..(7): Xaa is a standard amino acid or a non-genetically-encoded amino acid
  MISC_FEATURE (9)..(12): Xaa is a standard amino acid or a non-genetically-encoded amino acid
  MISC_FEATURE (18)..(18): Xaa is a standard amino acid or a non-genetically-encoded amino acid
LRRFSTXPXXXXDINDVXNF

SEQ ID NO: 3 - protein, 20 aa, Artificial Sequence
  Synthetic Peptide
LRRFSTAPFAFIDINDVINF

SEQ ID NO: 4 - protein, 20 aa, Artificial Sequence
  Synthetic Peptide
LRRFSTAPFAFININNVINF

SEQ ID NO: 5 - protein, 20 aa, Artificial Sequence
  Synthetic Peptide
LRRFSTAPFAFIDINDVINW

SEQ ID NO: 6 - protein, 9 aa, Artificial Sequence
  Synthetic Peptide
FTNINNVTN

SEQ ID NO: 7 - protein, 9 aa, Artificial Sequence
  Synthetic Peptide
FTDINDVTN

SEQ ID NO: 8 - protein, 10 aa, Artificial Sequence
  Synthetic Peptide
  MISC_FEATURE (1)..(1): Xaa is 4-chlorophenylalanine (4-ClPhe)
  MISC_FEATURE (2)..(2): Xaa is 2-Aminobutyric acid (Abu)
  MISC_FEATURE (8)..(8): Xaa is 2-Aminobutyric acid (Abu)
XXNINNVXNF

SEQ ID NO: 9 - protein, 10 aa, Artificial Sequence
  Synthetic Peptide
  MISC_FEATURE (2)..(2): Xaa is 2-Aminobutyric acid (Abu)
  MISC_FEATURE (8)..(8): Xaa is 2-Aminobutyric acid (Abu)
AXNINNVXNF

SEQ ID NO: 10 - protein, 9 aa, Artificial Sequence
  Synthetic Peptide
  MISC_FEATURE (2)..(2): Xaa is 2-Aminobutyric acid (Abu)
  MISC_FEATURE (8)..(8): Xaa is 2-Aminobutyric acid (Abu)
FXNINNVXN

SEQ ID NO: 11 - protein, 10 aa, Artificial Sequence
  Synthetic Peptide
  MISC_FEATURE (2)..(2): Xaa is Allylglycine (AllylGly)
  MISC_FEATURE (8)..(8): Xaa is Allylglycine (AllylGly)
FXNINNVXNF

SEQ ID NO: 12 - protein, 10 aa, Artificial Sequence
  Synthetic Peptide
FANINNVANF

SEQ ID NO: 13 - protein, 10 aa, Artificial Sequence
  Synthetic Peptide
FIDINDVINF

SEQ ID NO: 14 - protein, 10 aa, Artificial Sequence
  Synthetic Peptide
FIDINDVINW

SEQ ID NO: 15 - protein, 10 aa, Artificial Sequence
  Synthetic Peptide
FININNVINF

SEQ ID NO: 16 - protein, 10 aa, Artificial Sequence
  Synthetic Peptide
FSNINNVSNF

SEQ ID NO: 17 - protein, 10 aa, Artificial Sequence
  Synthetic Peptide
FVNINNVVNF

SEQ ID NO: 18 - protein, 23 aa, Artificial Sequence
  Synthetic Peptide
  MISC_FEATURE (1)..(1): Xaa is D-Leucine
  MISC_FEATURE (4)..(4): Xaa is D-Leucine
XRRXRRFSTAPFAFIDINDVINF

SEQ ID NO: 19 - protein, 20 aa, Artificial Sequence
  Synthetic Peptide
  MISC_FEATURE (1)..(1): Xaa is D-Leucine
  MISC_FEATURE (20)..(20): Xaa is D-Phenylalanine
XRRFSTAPFAFIDINDVINX

SEQ ID NO: 20 - protein, 20 aa, Artificial Sequence
  Synthetic Peptide
  MISC_FEATURE (10)..(10): Xaa is D-Alanine
LRRFSTAPFXFIDINDVINF

SEQ ID NO: 21 - protein, 20 aa, Artificial Sequence
  Synthetic Peptide
  MISC_FEATURE (20)..(20): Xaa is D-Alanine
LRRFSTAPFAFIDINDVINX

SEQ ID NO: 22 - protein, 20 aa, Artificial Sequence
  Synthetic Peptide
  MISC_FEATURE (10)..(10): Xaa is D-Alanine
LRRFSTAPFXFIDINDVINF

SEQ ID NO: 23 - protein, 20 aa, Artificial Sequence
  Synthetic Peptide
  MISC_FEATURE (12)..(12): Xaa is 2-Aminobutyric acid (Abu)
  MISC_FEATURE (18)..(18): Xaa is 2-Aminobutyric acid (Abu)
LRRFSTMPAMFXNINNVXNF

SEQ ID NO: 24 - protein, 20 aa, Artificial Sequence
  Synthetic Peptide
  MISC_FEATURE (10)..(10): Xaa is D-Alanine
LRRFSTMPFXFININNVINF

SEQ ID NO: 25 - protein, 20 aa, Artificial Sequence
  Synthetic Peptide
  MISC_FEATURE (10)..(10): Xaa is norleucine (Nle)
  MISC_FEATURE (12)..(12): Xaa is 2-Aminobutyric acid (Abu)
  MISC_FEATURE (18)..(18): Xaa is 2-Aminobutyric acid (Abu)
LRRFSTMPFXFXNINNVXNF

SEQ ID NO: 26 - protein, 20 aa, Artificial Sequence
  Synthetic Peptide
  MISC_FEATURE (12)..(12): Xaa is 2-Aminobutyric acid (Abu)
  MISC_FEATURE (18)..(18): Xaa is 2-Aminobutyric acid (Abu)
LRRFSTMPFAFXNINNVXNF

SEQ ID NO: 27 - protein, 20 aa, Artificial Sequence
  Synthetic Peptide
LRRFSTMPFAFININNVINF

SEQ ID NO: 28 - protein, 20 aa, Artificial Sequence
  Synthetic Peptide
  MISC_FEATURE (10)..(10): Xaa is D-Alanine
LRRFSTMPFXFININNVINF

SEQ ID NO: 29 - protein, 20 aa, Artificial Sequence
  Synthetic Peptide
  MISC_FEATURE (11)..(11): Xaa is 4-chlorophenylalanine (4-ClPhe)
  MISC_FEATURE (12)..(12): Xaa is 2-Aminobutyric acid (Abu)
  MISC_FEATURE (18)..(18): Xaa is 2-Aminobutyric acid (Abu)
LRRFSTMPFMXXNINNVXNF

SEQ ID NO: 30 - protein, 20 aa, Artificial Sequence
  Synthetic Peptide
  MISC_FEATURE (12)..(12): Xaa is 2-Aminobutyric acid (Abu)
  MISC_FEATURE (18)..(18): Xaa is 2-Aminobutyric acid (Abu)
LRRFSTMPFMAXNINNVXNF

SEQ ID NO: 31 - protein, 20 aa, Artificial Sequence
  Synthetic Peptide
  MISC_FEATURE (12)..(12): Xaa is 2-Aminobutyric acid (Abu)
  MISC_FEATURE (18)..(18): Xaa is 2-Aminobutyric acid (Abu)
LRRFSTMPFMFXNINNVXNF

SEQ ID NO: 32 - protein, 20 aa, Artificial Sequence
  Synthetic Peptide
  MISC_FEATURE (12)..(12): Xaa is Allylglycine (AllylGly)
  MISC_FEATURE (18)..(18): Xaa is Allylglycine (AllylGly)
LRRFSTMPFMFXNINNVXNF

SEQ ID NO: 33 - protein, 20 aa, Artificial Sequence
  Synthetic Peptide
LRRFSTMPFMFANINNVANF

SEQ ID NO: 34 - protein, 20 aa, Artificial Sequence
  Synthetic Peptide
LRRFSTMPFMFGNINNVGNF

SEQ ID NO: 35 - protein, 16 aa, Artificial Sequence
  Synthetic Peptide
LRRFSTMPFMFININN

SEQ ID NO: 36 - protein, 20 aa, Artificial Sequence
  Synthetic Peptide
LRRFSTMPFMFININNVINF

SEQ ID NO: 37 - protein, 20 aa, Artificial Sequence
  Synthetic Peptide
LRRFSTMPFMFSNINNVSNF

SEQ ID NO: 38 - protein, 16 aa, Artificial Sequence
  Synthetic Peptide
LRRFSTMPFMFTNINN

SEQ ID NO: 39 - protein, 20 aa, Artificial Sequence
  Synthetic Peptide
LRRFSTMPFMFTNINNVTNF

SEQ ID NO: 40 - protein, 20 aa, Artificial Sequence
  Synthetic Peptide
LRRFSTMPFMFVNINNVVNF

(19) United States
(12) Patent Application Publication
(10) Pub. No.: US 2020/0179285 A1
(43) Pub. Date: Jun. 11, 2020

(54) BIODEGRADABLE MICROPARTICLES FOR SUSTAINED DELIVERY OF ANTI-ANGIOGENIC PEPTIDE

(71) Applicants: The Johns Hopkins University, Baltimore, MD (US); AsclepiX Therapeutics, Inc., Baltimore, MD (US)

(72) Inventors: Jordan J. GREEN, Baltimore, MD (US); Niranjan PANDEY, Baltimore, MD (US); Aleksander S. POPEL, Baltimore, MD (US); Peter A. CAMPOCHIARO, Baltimore, MD (US); Jayoung KIM, Baltimore, MD (US); Raquel LIMA E. SILVA, Baltimore, MD (US); Ron SHMUELI, Baltimore, MD (US); Adam MIRANDO, Baltimore, MD (US)

(21) Appl. No.: 16/612,124

(22) PCT Filed: May 8, 2018
(86) PCT No.: PCT/US18/31663

§ 371 Date: Nov. 8, 2019

Related U.S. Application Data
(60) Provisional application No. 62/502,913, filed on May 8, 2017.

(51) Int. Cl. A61K 9/16; A61K 38/08; A61K 38/10; A61K 38/39; A61K 9/00

(52) U.S. Cl. CPC A61K 9/1647; A61K 38/08; A61K 9/0051; A61K 38/39; A61K 9/0024; A61K 38/10

(57) ABSTRACT

The present invention provides microparticle compositions comprising anti-angiogenic peptides, as well as methods of treatment, including for macular degeneration.

### CROSS-REFERENCE TO RELATED APPLICATIONS

This application claims the benefit of U.S. Provisional Application No. 62/502,913, filed May 8, 2017, the contents of which is incorporated herein by reference in its entirety.

### FEDERALLY SPONSORED RESEARCH OR DEVELOPMENT

This invention was made with government support under R21-EY023148 awarded by the National Institutes of Health (NIH). The government has certain rights in the invention.

### BACKGROUND

Age-Related Macular Degeneration (AMD) is currently a leading cause of vision loss. It affects more than 10 million Americans, which is more than cataracts and glaucoma combined. Wet AMD involves growth of new blood vessel in the choroid layer behind the retina. The new blood vessels tend to leak fluid, lipids, and blood. The leakage can cause scar tissue to form and retinal cells to stop functioning. Currently, the most common and effective clinical treatment for wet AMD is periodic intravitreal (into the eye) injection of an anti-angiogenesis drug. Such intravitreal injections are unpleasant, to say the least, which discourages patients' compliance, especially when the periodic intravitreal injection occurs frequently, such as monthly. Thus, there is a need for treatment of wet AMD and other ocular conditions that provide potent but extended effectiveness per treatment; thereby requiring less-frequent intravitreal injections.

In various aspects and embodiments, the present invention addresses these needs.

### SUMMARY

In various aspects and embodiments, the present invention provides microparticle compositions providing extended release of anti-angiogenic peptides, as well as methods of treatment, for age-related macular degeneration (AMD), e.g., wet AMD.

The microparticle of the present invention comprises poly(lactide-co-glycolide) (PLGA) having a having lactic acid (LA) to glycolic acid (GA) ratio (L/G) of more than 1:1; the microparticle further comprises, e.g., encapsulates, an anti-angiogenic peptide derived from the α5 fibril of type IV collagen. In embodiments, the PLGA is at least 60:40 L/G, e.g., 65:35 L/G, 75:25 L/G, and 85:15 L/G. In embodiments, the anti-angiogenic peptide has the amino acid sequence of any one of SEQ ID NO:1 to SEQ ID NO:40, e.g., LRRFSTXPXXXXNINNVXNF (SEQ ID NO:1), where X is a standard amino acid or a non-genetically-encoded amino acid, LRRFSTXPXXXXDINDVXNF (SEQ ID NO:2), where X is a standard amino acid or a non-genetically-encoded amino acid, LRRFSTAPFAFIDINDVINF (SEQ ID NO:3), or LRRFSTAPFAFININNVINF (SEQ ID NO:4). In embodiments, the microparticle is spherical or is non-spherical, e.g., has an ellipsoidal shape. In embodiments, the microparticle further comprises PLGA-PEG copolymers. In embodiments, a microparticle comprises from about 0.1% to about 10% peptide by weight of the microparticle, e.g., about 5% peptide by weight of the microparticle.

Aspects and embodiments of the present invention include pharmaceutical compositions comprising any of the herein-described microparticles and a pharmaceutically acceptable carrier or excipient. In embodiments, the pharmaceutical composition further comprises excess free peptide, e.g., selected from one or more of SEQ ID NO:1 to SEQ ID NO:40, e.g., LRRFSTXPXXXXNINNVXNF (SEQ ID NO:1), where X is a standard amino acid or a non-genetically-encoded amino acid, LRRFSTXPXXXXDINDVXNF (SEQ ID NO:2), where X is a standard amino acid or a non-genetically-encoded amino acid, LRRFSTAPFAFIDINDVINF (SEQ ID NO:3), or LRRFSTAPFAFININNVINF (SEQ ID NO:4). In embodiments, the free peptide and the peptide of the microparticle are the same peptide; alternately, the free peptide and the peptide of the microparticle are different peptides. In these embodiments, the pharmaceutical composition provides an initial effect from the one or more free peptides and a sustained effect over time through the controlled, extended release of the one or more peptides encapsulated in microparticles.

Aspects and embodiments of the present invention include methods for treating one or more of macular degeneration (e.g., age-related macular degeneration, “AMD”), macular edema (e.g., diabetic macular edema), retinal vein occlusion, and diabetic retinopathy. The methods comprise administering any of the herein-described pharmaceutical composition by intravitreal administration to a subject in need. In embodiments the AMD is “wet” AMD. In embodiments, the pharmaceutical composition is administered no more than once per month, e.g., no more than once per two months, no more than once per three months, and no more than once per four months. In embodiments, the subject's condition is refractory or only partially responsive to a VEGF blockade therapy.

Embodiments of the invention will now be described with reference to the Drawings and the following Detailed Description.

### BRIEF DESCRIPTION OF THE DRAWINGS

FIG. 1 shows preparation of poly(lactide-co-glycolide) (PLGA) particles encapsulating the AXT107 peptide (SEQ ID NO:3). Microparticles having a lactic acid (LA) to glycolic acid (GA) ratio (LA:GA ratio) of 65:35 (Low ratio lactic acid Microparticles; “LMP”) and 85:15 (High ratio lactic acid Microparticles; “HMP”) were prepared, either spherical or stretched (ellipsoidal).

FIG. 2A and FIG. 2B show scanning electron micrographs of 5% peptide-loaded, spherical LMP and 5% peptide-loaded, spherical HMP. These microparticles have an average diameter of about 5 microns (See FIG. 2C). FIG. 2D shows intact microparticles recovered from rabbit vitreous. FIG. 2E shows the cumulative release of peptide from LMP, Middle ratio lactic acid Microparticles (MMP), and HMP over 12 weeks.

FIG. 3 shows steps evaluating intravitreal administration of microparticles in a mouse model of choroidal neovascularization (CNV) or in a mouse model for subretinal neovascularization (subretinal NV) using transgenic rhoVEGF mice.

FIG. 4A shows that AXT107-containing microparticles were as effective as aflibercept (A) in inhibiting CNV, with the combination (A+AXT107) showing additional inhibition; “C” control. FIG. 4B shows that AXT107-containing microparticles promote CNV regression in a dose-dependent manner; “BL”, baseline. FIG. 4C and FIG. 4D show that AXT107-containing microparticles inhibit subretinal neovascularization in a dose-dependent manner (FIG. 4C) and reduce vascular leakage (FIG. 4D). FIG. 4E to FIG. 4G show AXT107-containing microparticles reduce VEGF-induced vascular leakage in rabbit eyes and show activity through day 60, when an aflibercept injection no longer shows activity.

FIG. 5A shows long-term in vivo efficacy of AXT107-containing HMP in inhibiting laser-induced CNV and FIG. 5B shows in vivo efficacy of AXT107-containing HMP in promoting regression following laser-induced CNV. FIG. 5C shows extended in vivo efficacy of AXT107-contining LMP in inhibiting laser-induced CNV and FIG. 5D shows in vivo efficacy of AXT107-contining LMP in promoting regression following laser-induced CNV. FIG. 5E and FIG. 5F show reduced vascular leakage in transgenic rhoVEGF mice following treatments with AXT107-contining microparticles.

FIG. 6A shows a scanning electron micrograph of ellipsoidal HMP. FIG. 6B shows in vivo efficacy of ellipsoidal HMP in inhibiting laser-induced CNV.

FIG. 7 shows AXT107 free peptide (50 μg) co-formulated with AXT107-encapsulated microparticles, injected intravitreally into rabbit eyes. The injection forms a depot that sits below the visual axis and does not obstruct vision.

### DETAILED DESCRIPTION

In various embodiments, the present invention provides microparticle compositions providing extended release of anti-angiogenic peptides, as well as methods of treatment, including for macular degeneration, macular edema, retinal vein occlusion, and diabetic retinopathy.

In some embodiments, the invention provides a microparticle comprising poly(lactide-co-glycolide) (PLGA) copolymers having more than 1:1 LA/GA ratio. The microparticles further comprise an anti-angiogenic peptide derived from the α5 fibril of type IV collagen. In various embodiments, the invention provides for greater duration of action upon intravitreal administration to a subject in need, including subjects that may be refractory or only partially-responsive to VEGF blockade or inhibitor therapy.

In some embodiments, the PLGA polymer is based on a LA/GA ratio (also referred to herein as “L/G ratio”) of from 55/45 to 95/5, such as: 60/40, 65/35, 70/30, 75/25, 80/20, 85/15, 90/10, or 95/5. PLGA degrades by hydrolysis of its ester linkages, and the LA/GA ratio helps tune the degradation rate in vivo. In some embodiments, polymers that are end-capped with esters (as opposed to the free carboxylic acid) provide for longer degradation half-lives. In embodiments, the PLGA polymer is based on a LA/GA ratio which is greater than 1/1.

In the below examples, microparticles having a higher L/G ratio (e.g., 85:15) tended to have longer peptide release relative to microparticles having a lower L/G ratio (e.g., 65:35). Microparticles having a higher L/G ratio provide a longer degradation half-live in vivo.

In some embodiments, the PLGA is at least 60:40 L/G. For example, the PLGA may be 65:35 L/G. In some embodiments, the PLGA is at least 75:25 L/G. For example, the PLGA may be about 85:15 L/G.

In some embodiments, the PLGA polymers used for fabricating microparticles have a molecular weight in the range of about 50 kDa to about 200 kDa, such as from 100 kDa to about 200 kDa. The microparticles may optionally further comprise poly (lactic-co-glycolic acid) polyethylene glycol (PLGA-PEG) block copolymers. In some embodiments, the microparticles comprise PLGA and PLGA-PEG polymers. For example, the microparticles may comprise from about 10% to about 95% PLGA-PEG copolymers (by mass of polymer), or in some embodiments, from 10% to 50%, or from 10% to 25%, or from 25% to 90%, or from 50% to 90%, or from 70% to 90%, or from 80% to 95% PLGA-PEG copolymer. For example, the microparticles may comprise from 10 to 25% PLGA-PEG copolymer, or from about 70% to about 90% PLGA-PEG copolymer, or about 60% to about 75% PLGA-PEG copolymer, or about 40% to about 60% PLGA-PEG copolymer, or about 20% to about 40% PLGA-PEG copolymer. Remaining polymers may be PLGA in various embodiments. In some embodiments, the PEG portion of the block copolymer may be in the range of about 1 kDa to about 5 kDa, or in some embodiments, about 1 kDa to about 3 kDa, or from about 3 kDa to about 5 kDa. In still other embodiments, the PEG portion of the block copolymers is from about 5 kDa to about 10 kDa, such as from about 5 kDa to about 8 kDa. In some embodiments, the microparticle has a size (average diameter or longest axis) within the range of about 500 nm to about 25 μm, or within the range of about 2 to about 20 μm, or from about 5 to about 15 μm. In some embodiments, the particles are spherical with an average diameter of from 3 to 6 μm. In some embodiments, the microparticle has a zeta potential in 10 mM NaCl that is negative within the range of about −5 mV to about −40 mV, and in some embodiments, from about −10 mV to about −30 mV (e.g., about −20, about −25, or about −30 mV). In other embodiments, the microparticle has a zeta potential in 10 mM NaCl that is approximately neutral between −10 mV to +10 mV, and in some embodiments, from about −5 mV to about +5 mV (e.g. about −4 or −2 mV).

In various aspects and embodiments, peptides and peptide agents disclosed herein are delivered in the form of microparticle formulations as described in U.S. Pat. Nos. 9,056,923, 9,802,984, and WO 2017/087825, which are hereby incorporated by reference in their entireties. For example, the peptides agents may be conjugated to the surface of and/or encapsulated within, the microparticle.

Microparticles of the present invention may encapsulate any peptide or peptide agent described herein.

Peptides derived from the α5 fibril of type IV collagen include those described in U.S. Pat. Nos. 9,056,923, 9,802,984, and WO 2017/087825, each of which is hereby incorporated by reference in its entirety.

In embodiments, the peptides target α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and platelet-derived growth factor receptor (PDGFR). In some embodiments, the peptide agent activates the Tie2 receptor kinase signaling pathway, which regulates vascular permeability.

In various embodiments, the peptide may comprise the amino acid sequence LRRFSTXPXXXNINNVXNF (SEQ ID NO:1) or LRRFSTXPXXXXDINDVXNF (SEQ ID NO:2), where X is a standard amino acid or non-genetically-encoded amino acid. In some embodiments, X at position 7 is M, A, or G; X at position 9 is F, A, Y, or G; X at position 10 is M, A, G, dA, or Nle; X at position 11 is F, A, Y, G, or 4-ClPhe; X at position 12 and position 18 are independently selected from Abu, G, S, A, V, T, I, L or Allyl-Gly. In various embodiments, the peptide contains about 30 amino acids or less, or about 25 amino acids of less, or about 24 amino acids, or about 23 amino acids, or about 22 amino acids, or about 21 amino acids, or about 20 amino acids. In still other embodiments, a total of from one to ten amino acids, such as one, two or three amino acids of SEQ ID NO:1 or 2 are deleted from one or more termini. Derivatives of the peptides include peptides having from 1 to 5 amino acid substitutions, insertions, or deletions (e.g., 1, 2, 3, 4, or 5 amino acid substitutions, insertions, or deletions collectively) with respect to SEQ ID NO:1 or 2. In some embodiments, the core sequence of DINDV or NINNV is maintained in the derivative. Amino acid substitutions can optionally be at positions occupied by an X at the corresponding position of SEQ ID NO: 1 or 2. The peptide generally has at least 8 amino acids. Exemplary peptides include peptides comprising the amino acid sequence LRRFSTAPFAFIDINDVINF (SEQ ID NO:3) or LRRFSTAPFAFININNVINF (SEQ ID NO:4).

Alternatively, the microparticles may comprise various derivatives of the peptides defined by SEQ ID NO:1 to SEQ ID NO:4, including LRRFSTAPFAFIDINDVINW (SEQ ID NO:5), FTNINNVTN (SEQ ID NO:6), or FTDINDVTN (SEQ ID NO:7).

In some embodiments, amino acid substitutions are independently selected from conservative or non-conservative substitutions. In these or other embodiments, the peptide includes from 1 to 10 amino acids added to one or both termini (collectively). The N-terminus and/or C-terminus may optionally be occupied by another chemical group (other than amine or carboxy, e.g., amide or thiol), and which can be useful for conjugation of other moieties, including PEG or PLGA-PEG copolymers. In some embodiments, the C-terminus is occupied by an amide group.

Conservative substitutions may be made, for instance, on the basis of similarity in polarity, charge, size, solubility, hydrophobicity, hydrophilicity, and/or the amphipathic nature of the amino acid residues involved. The 20 genetically encoded amino acids can be grouped into the following six standard amino acid groups:
(1) hydrophobic: Met, Ala, Val, Leu, Ile;
(2) neutral hydrophilic: Cys, Ser, Thr; Asn, Gln;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

In various embodiments, the peptide agent is a peptide of from about 8 to about 30 amino acids, or from about 10 to about 20 amino acids, and has at least 4, at least 5, or at least 6 contiguous amino acids of SEQ ID NO:2, 3, 4, 5, 6, or 7. In some embodiments, the peptide contains at least one, at least two, or at least three D-amino acids, e.g., dA, dL, and dF. In some embodiments, the peptide contains from one to about five (e.g., 1, 2, or 3) non-genetically encoded amino acids, which are optionally independently selected from 2-Aminobutyric acid (Abu), norleucine (Nle), 4-chlorophenylalanine (4-ClPhe), and Allylglycine (AllylGly). In some embodiments, the peptide is a retro-inverso peptide, based on an amino acid sequence described herein.

Exemplary peptide agents, which may be derivatives of the peptides of SEQ ID NO: 3 to SEQ ID NO:7 in accordance with the disclosure, include:

(SEQ ID NO: 8)
(4-ClPhe)(Abu)NINNV(Abu)NF,
(SEQ ID NO: 9)
A(Abu)NINNV(Abu)NF,
(SEQ ID NO: 10)
F(Abu)NINNV(Abu)N,
(SEQ ID NO: 11)
F(AllyGly)NINNV(AllyGly)NF,
(SEQ ID NO: 12)
FANINNVANF,
(SEQ ID NO: 13)
FIDINDVINF,
(SEQ ID NO: 14)
FIDINDVINW,
(SEQ ID NO: 15)
FININNVINF,
(SEQ ID NO: 16)
FSNINNVSNF,
(SEQ ID NO: 17)
FVNINNVVNF,
(SEQ ID NO: 18)
(dL)RR(dL)RRFSTAPFAFIDINDVINF,
(SEQ ID NO: 19)
(dL)RRFSTAPFAFIDINDVIN(dF),
(SEQ ID NO: 20)
LRRFSTAPF(dA)FIDINDVINF,
(SEQ ID NO: 21)
LRRFSTAPFAFIDINDVIN(dF),
(SEQ ID NO: 22)
LRRFSTAPFdAFIDINDVINF,
(SEQ ID NO: 23)
LRRFSTMPAMF(Abu)NINNV(Abu)NF,
(SEQ ID NO: 24)
LRRFSTMPF(dA)FININNVINF,
(SEQ ID NO: 25)
LRRFSTMPF(Nle)F(Abu)NINNV(Abu)NF,
(SEQ ID NO: 26)
LRRFSTMPFAF(Abu)NINNV(Abu)NF,
(SEQ ID NO: 27)
LRRFSTMPFAFININNVINF,
(SEQ ID NO: 28)
LRRFSTMPFdAFININNVINF,
(SEQ ID NO: 29)
LRRFSTMPFM(4-ClPhe)(Abu)NINNV(Abu)NF,
(SEQ ID NO: 30)
LRRFSTMPFMA(Abu)NINNV(Abu)NF,
(SEQ ID NO: 31)
LRRFSTMPFMF(Abu)NINNV(Abu)NF,
(SEQ ID NO: 32)
LRRFSTMPFMF(AllyGly)NINNV(AllyGly)NF,
(SEQ ID NO: 33)
LRRFSTMPFMFANINNVANF,
(SEQ ID NO: 34)
LRRFSTMPFMFGNINNVGNF,
(SEQ ID NO: 35)
LRRFSTMPFMFININN,
(SEQ ID NO: 36)
LRRFSTMPFMFININNVINF,
(SEQ ID NO: 37)
LRRFSTMPFMFSNINNVSNF,
(SEQ ID NO: 38)
LRRFSTMPFMFTNINN,
(SEQ ID NO: 39)
LRRFSTMPFMFTNINNVTNF,
or
(SEQ ID NO: 40)
LRRFSTMPFMFVNINNVVNF.

In certain embodiments, compositions disclosed herein include a free or encapsulated retroinverso peptide based on any amino acid sequence selected from SEQ ID NO:1 to SEQ ID NO:40. Retro-inverso peptides are linear peptides whose amino acid sequence is reversed and the α-center chirality of the amino acid subunits is inverted as well. These peptides are designed by including D-amino acids in the reverse sequence to help maintain side chain topology similar to that of the original L-amino acid peptide. Retro-inverso peptides maintain side chain topology similar to that of the original L-amino acid peptide, and also render the peptide more resistant to proteolytic degradation. In embodiments, a retro-inverso peptide has binding characteristics similar to its corresponding L-amino acid peptide; retro-inverso peptides mimic the shape of peptide epitopes, the protein-protein interactions, and/or protein-peptide interfaces of the corresponding L-amino acid peptide.

The peptides or peptide agents can be chemically synthesized and purified using well-known techniques, such as solid-phase synthesis. See U.S. Pat. No. 9,051,349, which is hereby incorporated by reference in its entirety.

While the microparticle is substantially spherical in some embodiments, the microparticle may optionally be non-spherical (e.g., ellipsoidal).

There are various physical and chemical properties that can affect how a material interacts with a biological system. In the case of microparticles, the choice of material, the size distribution, and the shape distribution of the particles are all critical parameters affecting the particles' activity. Both the size and shape of a particle can affect the way the particle interacts with various cells of the body. For example, the shape of the particle can affect how well various cell types can uptake the particle, where an ellipsoidal particle is usually more difficult for a cell to uptake than a spherical particle. Stretching the shape of the particles can therefore reduce unwanted uptake of particles, such as by the immune system cells, thereby extending the half-life of the particles in the body. Optimization of the activity of a particle based system can be achieved by tuning the size and shape distribution of the particles.

In some embodiments, a microparticle is stretched at a temperature from above the polymer transition temperature up to the polymer degradation temperature to form a non-spherical microparticle. In some embodiments, the microparticle is stretched at a temperature above but close to the polymer transition temperature. For example, in some embodiments, if the polymer transition temperature is about 60° C., the microparticle is stretched at a temperature above 60° C. to about 70° C. In some embodiments, if the polymer transition temperature is about 60° C., the microparticle is stretched at a temperature above 60° C. to about 80° C. As used herein, the “polymer transition temperature” is the temperature range where the polymer transitions from a hard material to a soft or rubber-like material. As used herein, the “polymer degradation temperature” is the temperature where the polymer begins to disintegrate. In some embodiments, the microparticle is stretched at a temperature from about 60° C. to about 90° C. to form an a non-spherical microparticle. In some embodiments, the microparticle is stretched at a temperature of about 60° C.

In some embodiments, the dimensions of the particle and/or process for stretching the particles in as disclosed in WO 2013/086500 and in WO 2016/164458, which are hereby incorporated by reference in its entirety.

In particular embodiments, the three-dimensional microparticle comprises a prolate ellipsoid, wherein the dimension (a) along the x-axis is greater than the dimension (b) along the y-axis, and wherein the dimension (b) along the y-axis is substantially equal to the dimension (c) along the z-axis, such that the prolate ellipsoid can be described by the equation a>b=c. In other embodiments, the ellipsoid is a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c. In yet other embodiments, the ellipsoid is an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c. The presently disclosed asymmetrical particles, however, do not include embodiments in which a=b=c.

In still other embodiments, the microparticle has an aspect ratio ranging from about 1.1 to about 5. In other embodiments, the aspect ratio has a range from about 5 to about 10. In some embodiments, the aspect ratio has a range from about 1.5 to about 3.5.

In some embodiments, the ellipsoidal particles are from about 8 to about 25 microns along their longest axis, or in some embodiments, about 10 to about 20 microns.

In some aspects, the pharmaceutical composition comprises a population of the microparticles, and a pharmaceutically acceptable carrier or excipient. In some embodiments, the pharmaceutical composition comprises the peptide at from 0.1 to about 20%, or from 0.1 to about 10% of the total weight of the microparticle. In some embodiments, the composition comprises the peptide at from about 1% to about 8%, or about 2% to about 8%, of the total weight of the microparticle. In some embodiments, the composition comprises peptide at from 1% to about 7%, or about 1% to about 5%, of the total weight of the microparticle. In some embodiments, the composition comprises peptide at from about 5% to about 15% of the total weight of the microparticle composition. In various embodiments, the composition comprises from about 50 μg to about 1 mg of peptide agent (including free and encapsulated peptide). For example, exemplary compositions comprise from 100 μg to about 500 μg of peptide, or in some embodiments, from about 100 μg to about 1 mg of peptide.

In some embodiments, the pharmaceutical composition further comprises excess free peptide, such as a peptide selected from any one of SEQ ID NO:1 to SEQ ID NO:40, e.g., SEQ ID NO:1, SEQ ID NO:2, and/or SEQ ID NO:3. In embodiments, a pharmaceutical composition comprises microparticles encapsulating one or more peptides selected from any one of SEQ ID NO:1 to SEQ ID NO:40 and one or more free peptides selected from any one of SEQ ID NO:1 to SEQ ID NO:40. In embodiments, the encapsulated peptides are the same as the free peptides. In embodiments, the encapsulated peptides are different from the free peptides. In these embodiments, the pharmaceutical composition provides an initial effect from the one or more free peptides and a sustained effect over time through the controlled, extended release of the one or more peptides encapsulated in microparticles. In embodiments, the composition comprises from about 0.01 mg to about 1 mg, or about 0.01 to about 0.1 mg of free peptide. In some embodiments, the composition comprises from about 0.02 mg to about 0.08 mg of free peptide.

In various embodiments, the amount of free peptide is from 20% to about 95% of the peptide in the composition (in moles). For example, exemplary compositions have from about 20% to about 90% free peptide, from about 20% to about 80% free peptide, or from about 20% to about 60% free peptide, or from about 20% to about 40% free peptide. In some embodiments, the composition has from about 40% to about 80% free peptide, or from about 40% to about 60% free peptide (e.g., about 50% free peptide). In some embodiments, the composition comprises from 50% to about 80% free peptide, or from 50% to about 70% free peptide. The remainder of the peptide is associated with the microparticles, e.g., encapsulation.

In other aspects, the invention provides a method for treating one or more of macular degeneration, macular edema, retinal vein occlusion and diabetic retinopathy. The method comprises administering the pharmaceutical composition described herein by intravitreal administration to a subject in need. For example, the subject may have age-related macular degeneration (AMD), such as wet or dry AMD. In some embodiments, the subject has diabetic macular edema. In some embodiments, the subject has retinal vein occlusion or diabetic retinopathy.

The peptide formulated as described targets α5β1 and αVβ3 integrins, and can inhibit signaling through multiple receptors, including vascular endothelial growth factor receptor (VEGFR), hepatocyte growth factor receptor (HGFR), insulin-like growth factor receptor (IGFR), and epidermal growth factor receptor (EGFR), as well as promoting activation of Tie2.

The compositions have a long duration of action. For example, the pharmaceutical composition may be administered monthly, or in some embodiments, about once every two months, or about once every three months, or about once every four months, or about once every five or six months. In some embodiments, the composition is administered about 2, 3, or 4 times per year. The compositions are generally administered by intravitreal injection.

In some embodiments, the subject has a condition that is refractory or only partially-responsive to VEGF blockade therapy. In some embodiments, the composition is administered after unsuccessful VEGF blockade therapy, or as an alternative to VEGF blockade therapy. In some embodiments, the compositions are administered in combination with VEGF blockade therapy. In some embodiments, the VEGF blockade therapy is aflibercept (e.g., EYLEA), or similar agent.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example, within plus or minus 10%.

This invention is further illustrated by the following non-limiting examples.

### EXAMPLES

### Example 1: Microparticles Having a Higher Lactic Acid to Glycolic Acid Ratio have Longer Peptide Release Relative to Microparticles Having a Lower Lactic Acid to Glycolic Acid Ratio

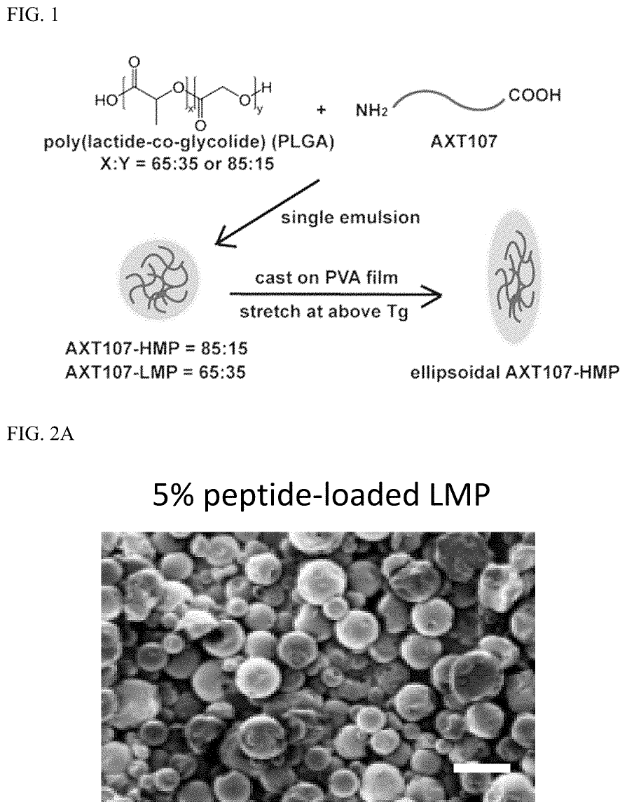

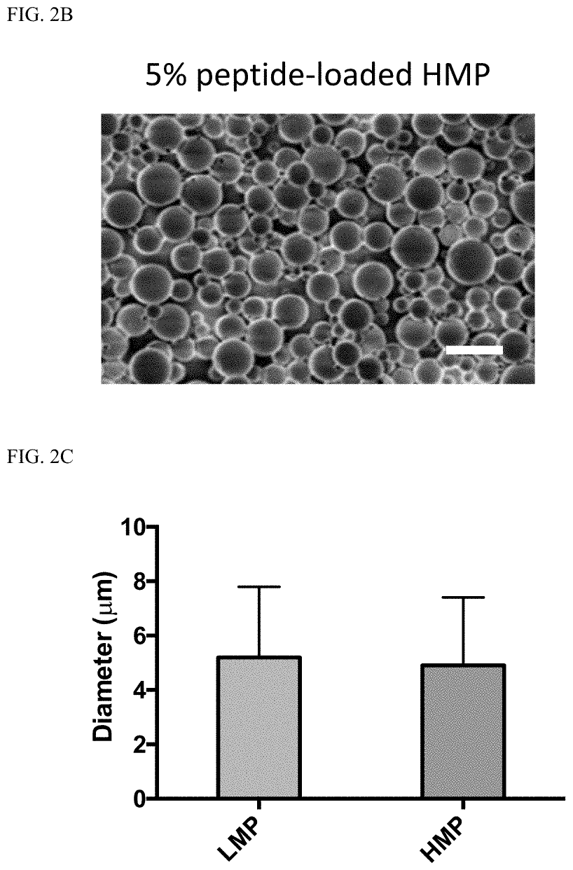

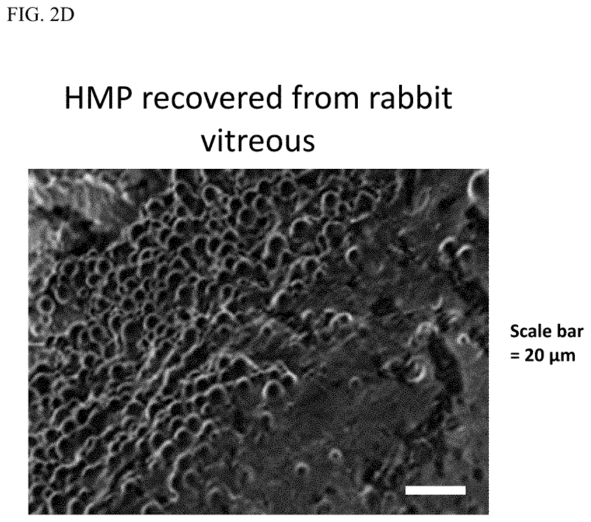

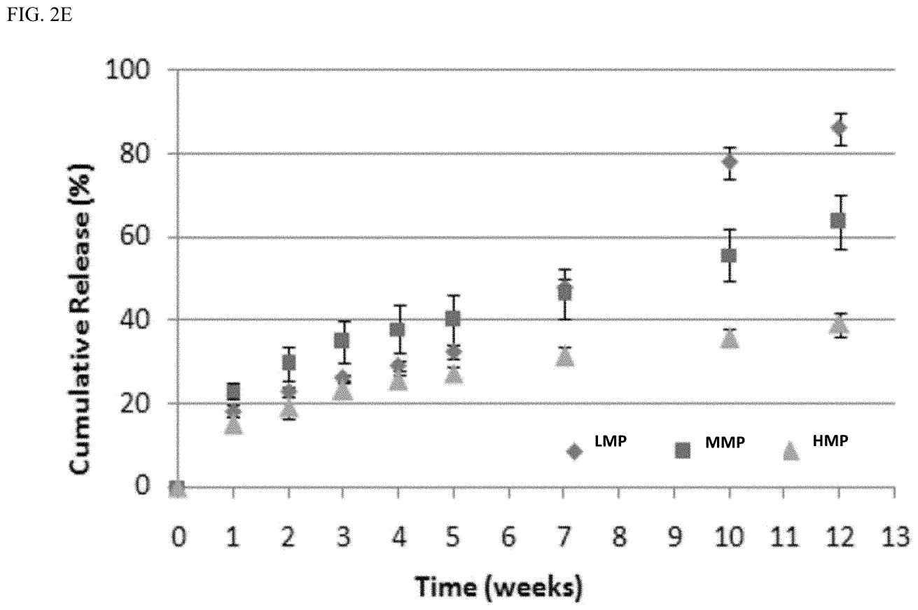

Microparticles comprising Poly(lactide-co-glycolide) “PLGA” were prepared with various lactic acid (LA) to glycolic acid (GA) ratios (LA:GA ratios) and loaded with the AXT107 peptide (which has the amino acid sequence of SEQ ID NO:3). As examples, low lactic acid microparticles (LMP) comprising a 65:35 LA:GA ratio, middle lactic acid microparticles (MMP) comprising a 75:25 LA:GA ratio, and high lactic acid microparticles (HMP) comprising a 85:15 LA:GA ratio were evaluated in this example; see FIG. 1. When not stretched at temperatures above its polymer transition temperature (see FIG. 1), 5% peptide-loaded, LMP and HMP were found to be roughly spherical (See FIG. 2A and FIG. 2B) and to have average diameter of about 5 microns (See FIG. 2C).

To initially test structural stability of microparticles in vivo, HMP were injected into a rabbit's eye and were later recovered. As shown in FIG. 2D, the microparticles recovered from rabbit vitreous were intact and retained their shape.

The peptide-release profiles of the microparticles were compared. For this, LMP, MMP, and HMP encapsulating the AXT107 peptide were incubated in a physiological saline and the cumulative amount of peptide released was measured at regular intervals. As shown in FIG. 2E, in the first week, the amount of peptide released was roughly similar for the three types of microparticles. However, by the second week, more loaded peptide had been released from the LMP when compared to the higher LA:GA ratio microparticles. At the end of the experiment, at twelve weeks, about 90% of the originally-loaded peptide was released from the LMP, whereas only about 40% of the originally-loaded peptide was released from the MMP.

These data show that microparticles having a higher LA:GA ratio provide longer-term release of encapsulated peptides relative to microparticles having a lower LA:GA ratio. Thus, microparticles having the higher LA:GA ratio may be used when longer-term release of a peptide is desired.

### Example 2: AXT107-Containing Microparticles Reduce Neovascularization and Vascular Leakage In Vivo

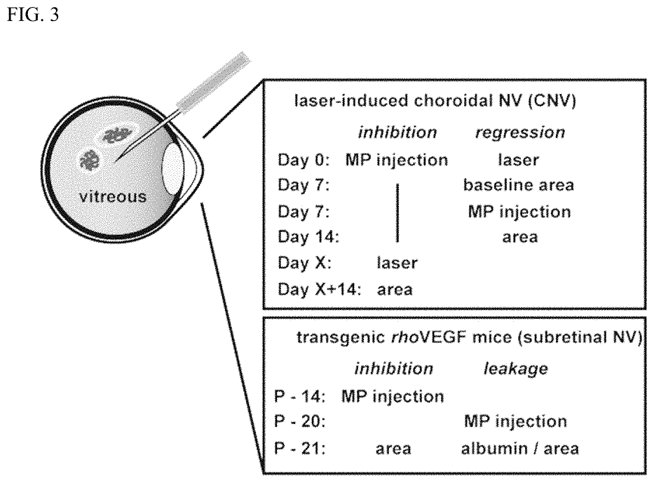

The ability of AXT107-containing microparticles to treat and/or reduce symptoms of choroidal neovascularization (CNV) and subretinal neovascularization (subretinal NV) was evaluated. CNV and subretinal NV are associated with age-related macular degeneration (AMD) and, particularly, “wet” AMD. Here, mouse models for CNV or subretinal NV were evaluated essentially as illustrated in FIG. 3.

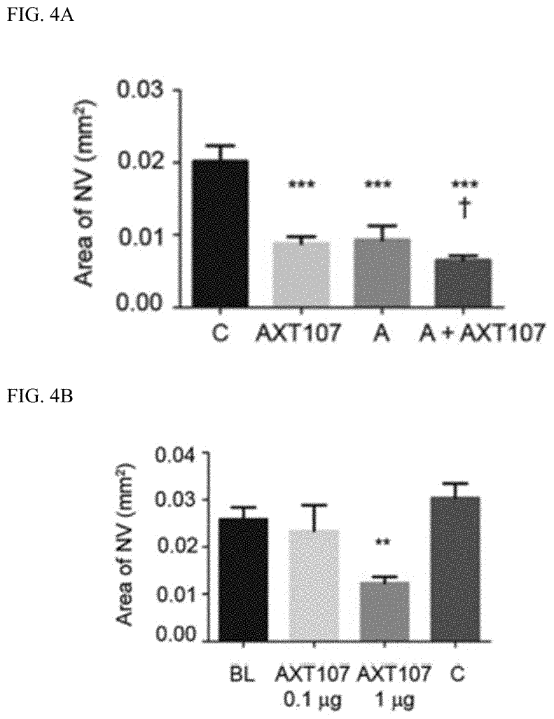

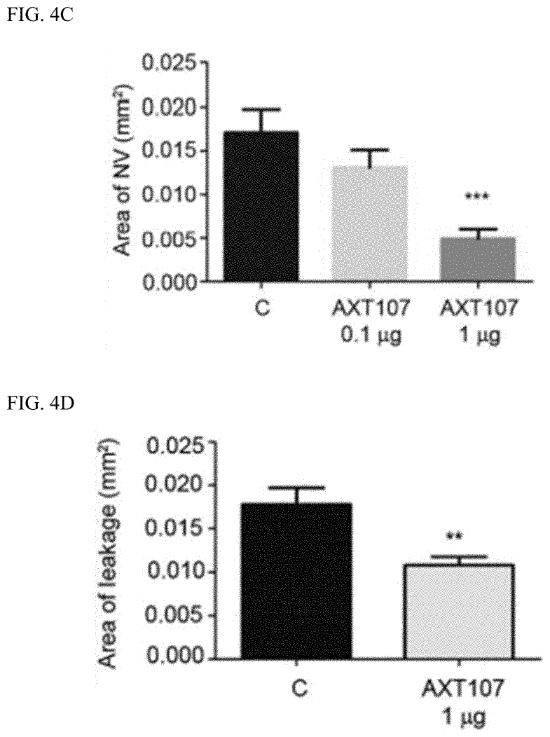

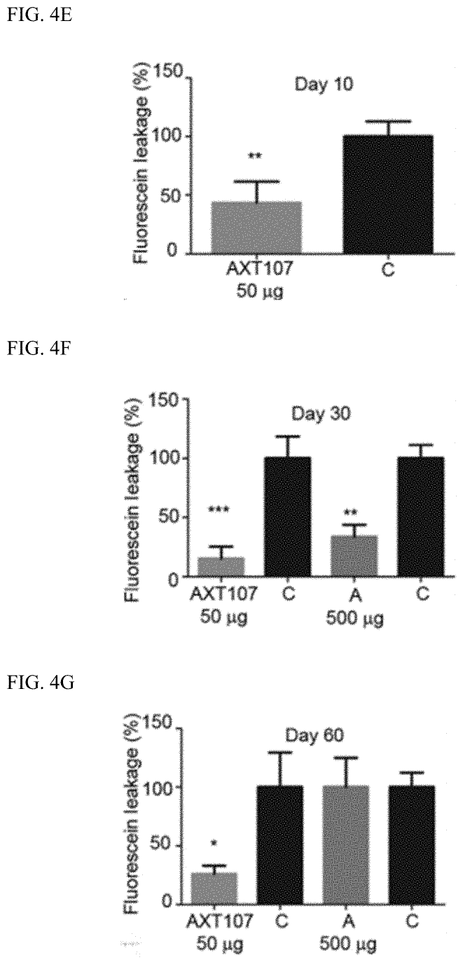

As shown in FIG. 4A, AXT107-containing microparticles were as effective as aflibercept (an FDA-approved treatment for wet macular degeneration; A) in inhibiting laser-induced CNV. Notably, the combination of aflibercept and AXT107-containing microparticles (A+AXT107) showed additional inhibition over either treatment alone. FIG. 4B shows that AXT107-containing microparticles promote CNV regression in a dose-dependent manner.

Similarly, as shown in FIG. 4C, the AXT107-containing microparticles were effective, in a dose-dependent manner, in inhibiting subretinal NV and were effective in reducing vascular leakage; see, FIG. 4D.

In this model, AXT107-containing microparticles reduced VEGF-induced vascular leakage (see, FIG. 4E to FIG. 4G). At 30 days after initial treatment, the AXT107-containing microparticles had greater effectiveness than aflibercept; see FIG. 4F. Moreover, as shown in FIG. 4G, a single injection of the AXT107-containing microparticles showed activity through day 60 whereas the aflibercept injection no longer showed activity at that time point.

These data show that AXT107-containing microparticles have activity against neovascularization and vascular leakage which is at least as effective as aflibercept; however, this activity persists beyond when aflibercept ceases to be active. Thus, treatments with AXT107-containing microparticles would require less frequent administration, i.e., intravitreal injection, than treatments with aflibercept.

### Example 3: AXT107-Containing HMP Provide Longer-Lasting In Vivo Effectiveness than AXT107-Containing LMP

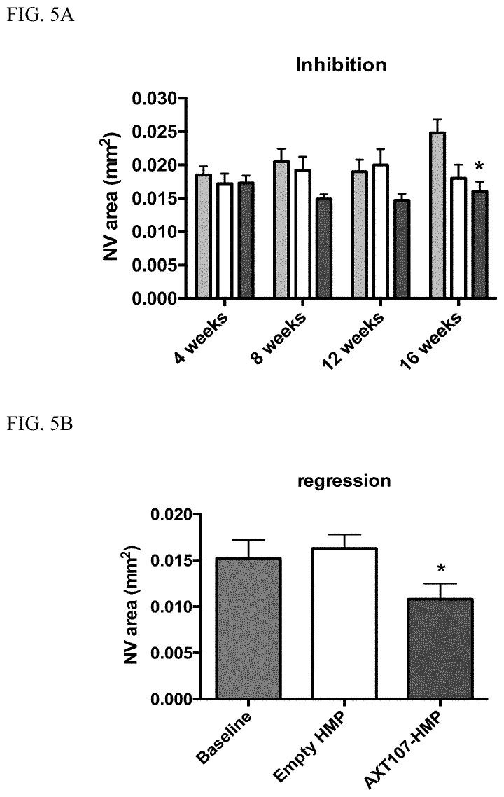

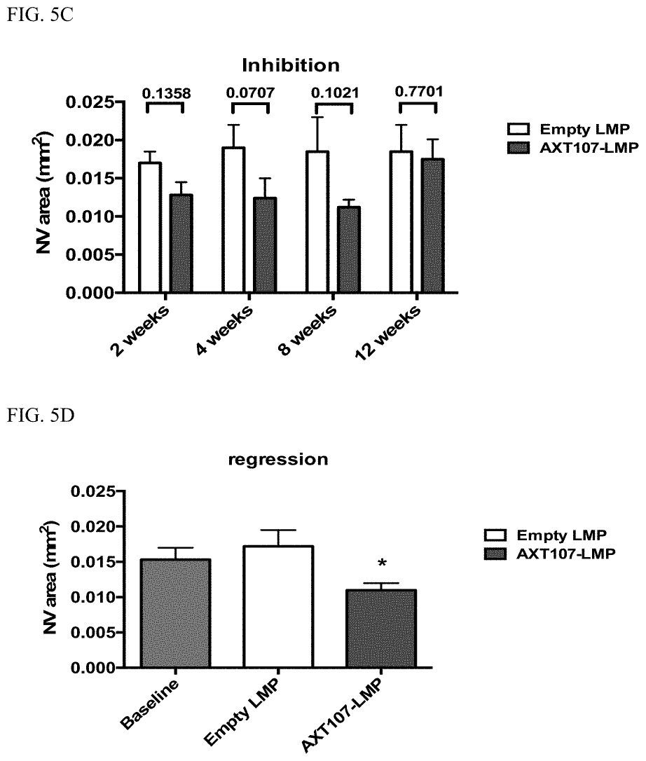

As shown in FIG. 5A, AXT107-containing HMP provided long-term (at least until 16 weeks after injection) in vivo efficacy in inhibiting laser-induced CNV; they promoted in vivo regression of CNV following laser-induction; see FIG. 5B. As shown in FIG. 5C, AXT107-containing LMP provided extended (at least until 8 weeks after injection) in vivo efficacy in inhibiting laser-induced CNV. At 12 weeks post injection, there appeared to be little difference between the AXT107-containing LMP and an empty LMP. Like the AXT107-containing HMP, the AXT107-containing LMP promoted in vivo regression following laser-induced; see FIG. 5D.

Finally, AXT107-containing HMP were effective in treating vascular leakage in transgenic rhoVEGF mice (a mouse model for subretinal NV); see FIG. 5E and FIG. 5F.

These data show that microparticles having a higher LA:GA ratio provide longer-term effectiveness in inhibiting laser-induced CNV and promoting in vivo regression of CNV relative to microparticles having a lower LA:GA ratio. Thus, microparticles having the higher LA:GA ratio may be useful when prolonged release of a peptide is desired.

### Example 4: Non-Spherical AXT107-Containing Microparticles are Effective in Treating In Vivo Laser-Induced CNV

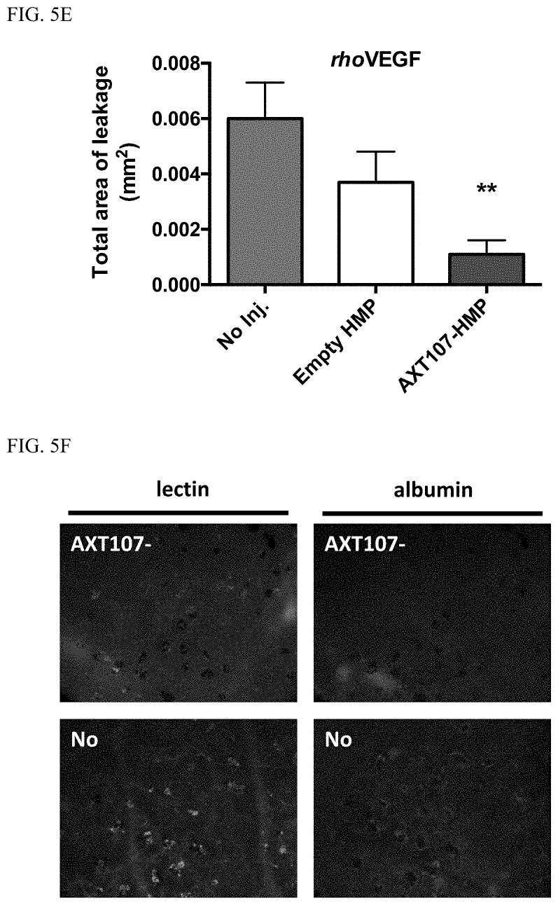

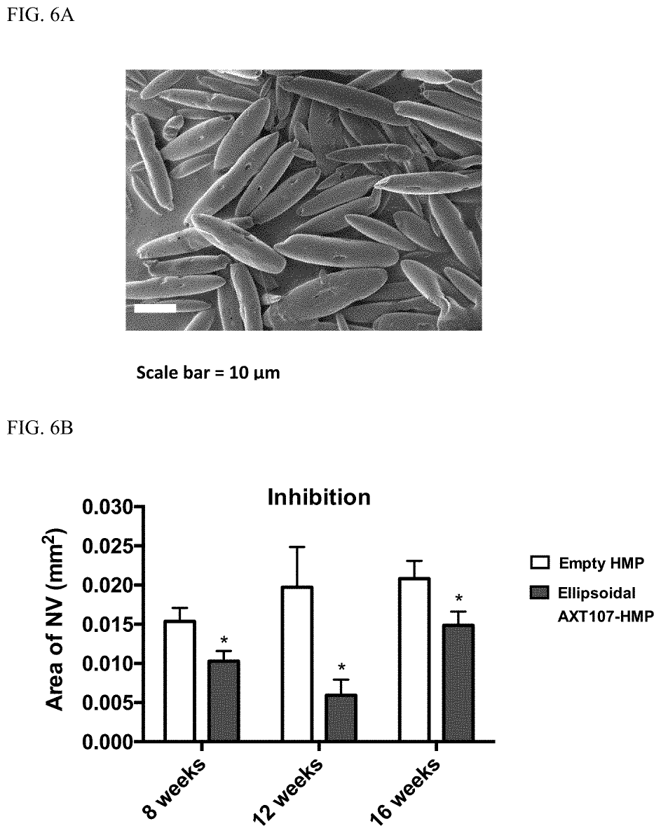

As shown in FIG. 1, AXT107-containing HMP were prepared and stretched into a non-spherical, e.g., ellipsoidal, shape. FIG. 6A is a scanning electron micrograph showing such non-spherical HMP.

As shown in FIG. 6B, the non-spherical AXT107-containing HMP provided strong (at least 12 weeks after injection) in vivo efficacy in inhibiting laser-induced CNV. At 16 weeks, activity of HMP remains measurable.

These data show that the shape of a microparticle may affect the rate at which a peptide cargo is released from a microparticle.

### Example 5: Formulation of AXT107 MP with Excess Free Peptide

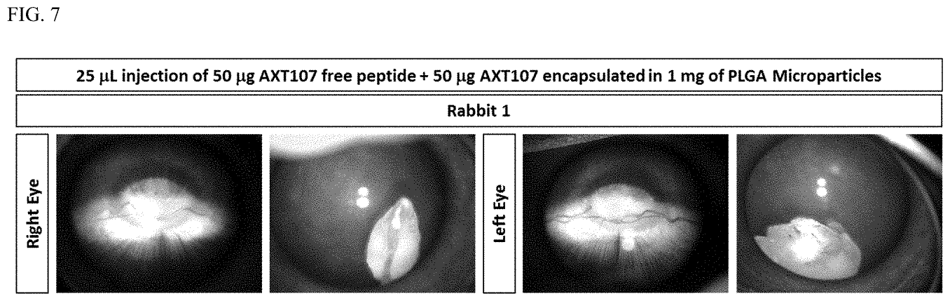

25 microliters of AXT107 free peptide (50 μg) plus AXT107 encapsulated in microparticles (50 μg in 1 mg of microparticles) was injected intravitreally into rabbit eyes. As demonstrated in FIG. 7, the injection forms a depot that sits below the visual axis and does not obstruct vision. The depot does not cause inflammation and enables controlled release over time of the encapsulated peptide.

These examples demonstrate that the anti-angiogenic peptide (e.g., AXT107) inhibited and promoted regression of choroidal neovascularization (CNV) in mice, inhibited and promoted regression of subretinal neovascularization in mice, and suppressed vascular leakage in mice and rabbits. Peptide-loaded microparticles were formulated with PLGA having different lactic acid to glycolic acid ratios; when compared to low lactic acid microparticles, high lactic acid microparticles (HMP) showed greater inhibition and promotion of regression of CNV at time points beyond two months after initial treatment. By heating at a temperature from above the polymer transition temperature up to the polymer degradation temperature, spherical peptide-loaded particles were physically transformed into non-spherical (e.g., ellipsoidal) shapes; the non-spherical microparticles had properties different from those of spherical microparticles. Additionally, non-spherical HMP showed significant CNV inhibition longer than spherical HMP. Finally, pharmaceutical compositions comprising encapsulated and free peptide form a depot when intravitreally injected; this depot which does not obstruct vision nor causes inflammation, yet enables controlled release over time of the encapsulated peptide.

1. A microparticle comprising poly(lactide-co-glycolide) (PLGA) having a having lactic acid (LA) to glycolic acid (GA) ratio (L/G) of more than 1:1, the microparticle further comprising an anti-angiogenic peptide derived from the α5 fibril of type IV collagen.

2. The microparticle of claim 1, wherein the PLGA is at least 60:40 L/G.

3. The microparticle of claim 2, wherein the PLGA is 65:35 L/G.

4. The microparticle of claim 2, wherein the PLGA is 75:25 L/G.

5. The microparticle of claim 2, wherein the PLGA is 85:15 L/G.

6. The microparticle of any one of claims 1 to 5, wherein the anti-angiogenic peptide has the amino acid sequence LRRFSTXPXXXXNINNVXNF (SEQ ID NO:1), where X is a standard amino acid or a non-genetically-encoded amino acid.

7. The microparticle of any one of claims 1 to 5, wherein the anti-angiogenic peptide has the amino acid sequence LRRFSTXPXXXXDINDVXNF (SEQ ID NO:2), where X is a standard amino acid or non-genetically-encoded amino acid.

8. The microparticle of claim 6 or 7, wherein the anti-angiogenic peptide is LRRFSTAPFAFIDINDVINF (SEQ ID NO:3) or LRRFSTAPFAFININNVINF (SEQ ID NO:4).

9. The microparticle of any one of claims 1 to 5, wherein the anti-angiogenic peptide is any one of:
(SEQ ID NO: 5)
LRRFSTAPFAFIDINDVINW,
(SEQ ID NO: 6)
FTNINNVTN,
(SEQ ID NO: 7)
FTDINDVTN,
(SEQ ID NO: 8)
(4-ClPhe)(Abu)NINNV(Abu)NF,
(SEQ ID NO: 9)
A(Abu)NINNV(Abu)NF,
(SEQ ID NO: 10)
F(Abu)NINNV(Abu)N,
(SEQ ID NO: 11)
F(AllyGly)NINNV(AllyGly)NF,
(SEQ ID NO: 12)
FANINNVANF,
(SEQ ID NO: 13)
FIDINDVINF,
(SEQ ID NO: 14)
FIDINDVINW,
(SEQ ID NO: 15)
FININNVINF,
(SEQ ID NO: 16)
FSNINNVSNF,
(SEQ ID NO: 17)
FVNINNVVNF,
(SEQ ID NO: 18)
(dL)RR(dL)RRFSTAPFAFIDINDVINF,
(SEQ ID NO: 19)
(dL)RRFSTAPFAFIDINDVIN(dF),
(SEQ ID NO: 20)
LRRFSTAPF(dA)FIDINDVINF,
(SEQ ID NO: 21)
LRRFSTAPFAFIDINDVIN(dF),
(SEQ ID NO: 22)
LRRFSTAPFdAFIDINDVINF,
(SEQ ID NO: 23)
LRRFSTMPAMF(Abu)NINNV(Abu)NF,
(SEQ ID NO: 24)
LRRFSTMPF(dA)FININNVINF,
(SEQ ID NO: 25)
LRRFSTMPF(Nle)F(Abu)NINNV(Abu)NF,
(SEQ ID NO: 26)
LRRFSTMPFAF(Abu)NINNV(Abu)NF,
(SEQ ID NO: 27)
LRRFSTMPFAFININNVINF,
(SEQ ID NO: 28)
LRRFSTMPFdAFININNVINF,
(SEQ ID NO: 29)
LRRFSTMPFM(4-ClPhe)(Abu)NINNV(Abu)NF,
(SEQ ID NO: 30)
LRRFSTMPFMA(Abu)NINNV(Abu)NF,
(SEQ ID NO: 31)
LRRFSTMPFMF(Abu)NINNV(Abu)NF,
(SEQ ID NO: 32)
LRRFSTMPFMF(AllyGly)NINNV(AllyGly)NF,
(SEQ ID NO: 33)
LRRFSTMPFMFANINNVANF,
(SEQ ID NO: 34)
LRRFSTMPFMFGNINNVGNF,
(SEQ ID NO: 35)
LRRFSTMPFMFININN,
(SEQ ID NO: 36)
LRRFSTMPFMFININNVINF,
(SEQ ID NO: 37)
LRRFSTMPFMFSNINNVSNF,
(SEQ ID NO: 38)
LRRFSTMPFMFTNINN,
(SEQ ID NO: 39)
LRRFSTMPFMFTNINNVTNF,
or
(SEQ ID NO: 40)
LRRFSTMPFMFVNINNVVNF.

10. The microparticle of any one of claims 1 to 9, wherein the microparticle is approximately spherical.

11. The microparticle of any one of claims 1 to 9, wherein the microparticle is non-spherical, e.g., is ellipsoidal.

12. The microparticle of any one of claims 1 to 11, further comprising PLGA-PEG copolymers.

13. The microparticle of claim 12, comprising from about 10% to about 95% PLGA-PEG copolymers (by mass of polymer).

14. The microparticle of any one of claims 1 to 13, wherein the microparticle comprises from about 0.1% to about 20% peptide by weight of the microparticle.

15. A pharmaceutical composition comprising the microparticle of any one of claims 1 to 14, and a pharmaceutically acceptable carrier or excipient.

16. The pharmaceutical composition of claim 15, further comprising excess free peptide, optionally being a peptide selected from SEQ ID NO:1 or SEQ ID NO:2, where X is a standard amino acid or non-genetically-encoded amino acid.

17. The pharmaceutical composition of claim 15, further comprising excess free peptide selected from SEQ ID NO:3 to SEQ ID NO: 40.

18. The pharmaceutical composition of claim 17, wherein the composition comprises from about 20% to about 95% free peptide (in moles).

19. The pharmaceutical composition of claim 17 or claim 18, wherein the anti-angiogenic peptide comprised by the microparticle is the same peptide as the free peptide.

20. The pharmaceutical composition of claim 17 or claim 18, wherein the anti-angiogenic peptide comprised by the microparticle is a different peptide from the free peptide.

21. A method for treating one or more of macular degeneration, macular edema, retinal vein occlusion, and diabetic retinopathy, comprising: administering the pharmaceutical composition of any one of claims 15 to 20 by intravitreal administration to a subject in need.

22. The method of claim 21, wherein the subject has an age-related macular degeneration (AMD).

23. The method of claim 22, wherein the AMD is wet AMD.

24. The method of claim 21, wherein the subject has diabetic macular edema.

25. The method of claim 21, wherein the subject has retinal vein occlusion or diabetic retinopathy.

26. The method of any one of claims 21 to 25, wherein the pharmaceutical composition is administered no more than once per month.

27. The method of claim 26, wherein the pharmaceutical composition is administered no more than once every two months.

28. The method of claim 27, wherein the pharmaceutical composition is administered no more than once every three months.

29. The method of claim 28, wherein the pharmaceutical composition is administered no more than once every four months.

30. The method of claim 28, wherein the pharmaceutical composition is administered no more than once every five or six months.

31. The method of any one of claims 21 to 30, wherein the subject's condition is refractory or only partially responsive to a VEGF blockade therapy.

32. The method of claim 31, wherein the pharmaceutical composition is administered instead of a VEGF blockade therapy.

33. The method of claim 31, wherein the pharmaceutical composition is administered to a patient undergoing a VEGF blockade therapy.
